(19) United States
(12) Patent Application Publication
(10) Pub. No.: US 2012/0289812 A1
(43) Pub. Date: Nov. 15, 2012

(54) APPARATUS FOR ACQUIRING BIOFUNCTIONAL INFORMATION, METHOD FOR ACQUIRING BIOFUNCTIONAL INFORMATION, AND PROGRAM THEREFOR

(75) Inventor: Takuji Oishi, Kyoto-shi (JP)

(73) Assignee: CANON KABUSHIKI KAISHA, Tokyo (JP)

(21) Appl. No.: 13/515,037

(22) PCT Filed: Jan. 31, 2011
(86) PCT No.: PCT/JP2011/052453

§ 371 Date: Jun. 11, 2012

(30) Foreign Application Priority Data
Feb. 4, 2010 (JP) 2010-022892
Jan. 21, 2011 (JP) 2011-010534

(51) Int. Cl. A61B 6/00

(52) U.S. Cl. 600/407

(57) ABSTRACT

An adverse effect of position displacement during measurements in calculating oxygen saturation can be decreased with an apparatus having: an acoustic wave detector, for receiving acoustic waves inside a subject, and for converting the acoustic waves to signals; and a processing apparatus for deriving biofunctional information using profiles of absorption coefficient derived from the signals, in which the processing apparatus includes: a first unit for deriving, from signals corresponding to light of first wavelength, first data showing a profile of first absorption coefficient, and from signals corresponding to light of second wavelength, second data showing a profile of second absorption coefficient; and a second unit for deriving the biofunctional information using the first and second data, and in which the second data has lower image spatial resolution than the first data.

### TECHNICAL FIELD

The present invention relates to an apparatus for acquiring biofunctional information, a method for acquiring biofunctional information, and a program for implementing the method.

### BACKGROUND ART

Imaging apparatuses using one of X-rays and ultrasound are used in many fields requiring nondestructive testing, such as the medical field. Particularly in the medical field, diagnosis using ultrasound echo involves an advantage of being noninvasive and thus is used in many situations. It is important to derive biofunctional information within a living body, that is, physiological information, for discovery of a disease site, such as a cancer. But in conventional diagnosis using X-ray or ultrasound echo, only shape information within a living body is derived. Therefore, Photoacoustic Tomography (PAT), one of the light imaging techniques, is proposed as a new noninvasive diagnosis method that can image biofunctional information.

In PAT, in vivo information is imaged by irradiating a subject with pulsed light generated from a light source and detecting an acoustic wave (typically ultrasound) which is generated from a living body tissue absorbing the energy of the light propagated and which is diffused within the subject. Information related to optical properties inside the subject can be made three-dimensionally visible by detecting a temporal change in acoustic waves received at a plurality of places surrounding the subject, and mathematically analyzing (that is, reconstructing) the derived signals. When a profile of initial pressure generation within the subject is detected by using this method, information on a profile of optical properties, such as a profile of light absorption coefficient, can be derived.

Examples of the detection of biofunctional information using PAT include measurement of oxygen saturation.

Oxygen saturation is content of hemoglobin bound to oxygen with respect to an amount of total hemoglobin in blood. Whether cardiopulmonary function operates normally or not can be measured by detecting oxygen saturation. In addition, oxygen saturation is an indicator for distinguishing the benignancy/malignancy of a tumor, and therefore is expected as a measure for efficient discovery of a malignant tumor.

Near-infrared light is used for the measurement of oxygen saturation. Near-infrared light has the property of being easily transmitted through water which constitutes a large portion of a living body, while being easily absorbed by hemoglobin in blood. Hemoglobin in a living body includes two states: deoxyhemoglobin not bound to oxygen and oxyhemoglobin bound to oxygen, and the optical absorption spectra in the respective states are different. Therefore, oxygen saturation can be found by performing measurement a plurality of times using pulsed lights having different wavelengths in the near-infrared region, and subjecting calculated light absorption coefficients to comparison operation. In other words, when a living body is irradiated with near-infrared light, oxygen saturation as a biofunctional information can also be imaged in addition to a blood vessel image as a shape information of the living body.

In acquiring biofunctional information by this method, however, it is necessary to subject the results performed for the same place in a plurality of measurements to comparison operation, and thus when the measurement positions do not match due to the movement of the body and the like, a misdirected result may be derived.

For the problem of the comparison of a plurality of measurements, such a technique as disclosed in Patent Literature 1 has been mentioned. In the technique of Patent Literature 1, a moving vector between images, measured for a particular region in the images, is extracted. Then, an adjustment such as zooming, rotation, and shift, of the image is performed based on the vector to correct position displacement (i.e. position adjustment), and the plurality of images are compared.

### CITATION LIST

### Patent Literature

PTL 1: Japanese Patent Application Laid-Open No. 2007-215930

### SUMMARY OF INVENTION

### Technical Problem

However, the position adjustment between images still remains problems as shown below.

A first problem is that the extraction of a moving vector involves low robustness. In the position adjustment between images, a point or a structure (referred to as characteristic structures) presumed to be the same place is found out on a plurality of images to be compared, and a moving vector is extracted based on the point or the structure. However, since a living body is elastic and deforms in a complicated manner, even if a characteristic structure can be identified, due to deformation thereof, the characteristic structure may not be extracted in another image. In addition, when no characteristic structure can be identified in the images, the extraction of a moving vector would become more difficult.

A second problem is that it is difficult to completely match all pixels. A moving vector is derived only with a representative point, such as a characteristic structure, and therefore, an interpolation is necessary for adjustment of the positions of the other regions. However, since a living body is elastic, it is difficult to adjust the position between a plurality of images pixel by pixel in the interpolated regions.

In view of the above problems, it is an object of the present invention to provide a technique that can acquire biofunctional information, such as oxygen saturation, with which position adjustment between images is not necessary even if position displacement occurs in comparing the results of a plurality of measurements.

### Solution to Problem

In an aspect of the present invention, the apparatus for acquiring biofunctional information, comprising: an acoustic wave detector, for receiving a plurality of acoustic waves generated when a subject is irradiated with a plurality of lights having different wavelengths, and for converting the plurality of acoustic waves to a plurality of signals corresponding to the plurality of lights; and a processing apparatus for deriving biofunctional information inside the subject using a plurality of profiles of absorption coefficient which are derived from the plurality of signals and are respectively corresponding to the plurality of signals, in which the processing apparatus includes: a first unit for deriving, from a signal corresponding to light having a first wavelength, first data showing a profile of first absorption coefficient corresponding to the light having the first wavelength, and deriving, from a signal corresponding to light having a second wavelength different from the first wavelength, second data showing a profile of second absorption coefficient corresponding to the light having the second wavelength; and a second unit for deriving the biofunctional information using the first data and the second data, and wherein the second data has lower image spatial resolution than the first data.

In another aspect of the present invention, the method for acquiring biofunctional information by: receiving acoustic waves generated when a subject is irradiated with a plurality of lights having different wavelengths, and converting the acoustic waves to a plurality of signals corresponding to the plurality of lights, by an acoustic wave detector; and deriving biofunctional information using a plurality of profiles of absorption coefficient which are calculated from the plurality of signals and are corresponding to the plurality of signals, includes the steps of: deriving, from an acoustic wave generated when the subject is irradiated with light having a first wavelength, first data showing a profile of first absorption coefficient corresponding to the light having the first wavelength; deriving, from an acoustic wave generated when the subject is irradiated with light having a second wavelength, second data showing a profile of second absorption coefficient corresponding to the light having the second wavelength, and having lower image spatial resolution than the first data; and deriving the biofunctional information using the first data and the second data.

In yet another aspect of the present invention, the program for allowing a computer to execute each step of a method for acquiring biofunctional information includes executing the steps of: deriving, from an acoustic wave generated when the subject is irradiated with light having a first wavelength, first data showing a profile of first absorption coefficient corresponding to the light having the first wavelength; deriving, from an acoustic wave generated when the subject is irradiated with light having a second wavelength, second data showing a profile of second absorption coefficient corresponding to the light having the second wavelength, and having lower image spatial resolution than the first data; and deriving the biofunctional information using the first data and the second data.

### Advantageous Effects of Invention

With the apparatus and the method for acquiring biofunctional information according to the present invention, oxygen saturation can be calculated with a minor error even if the position displacement of a subject occurs during measurements.

Further features of the present invention will become apparent from the following description of exemplary embodiments with reference to the attached drawings.

### BRIEF DESCRIPTION OF DRAWINGS

FIG. 1 is a schematic diagram illustrating the configuration of an apparatus according to one embodiment of the present invention.

FIG. 2 is a schematic diagram illustrating the flow of the data processing of the apparatus according to one embodiment of the present invention.

FIG. 3 is a flow chart illustrating the operation of the apparatus according to one embodiment of the present invention.

FIGS. 4A, 4B and 4C are schematic diagrams illustrating the concept of the present invention.

FIG. 5 is a schematic diagram illustrating the flow of the data processing of the apparatus according to one embodiment of the present invention.

FIG. 6 is a diagram illustrating oxygen saturation when position displacement does not occur.

FIG. 7 is a diagram illustrating oxygen saturation when position displacement occurs.

FIG. 8 is a diagram of oxygen saturation calculated by applying the present invention when position displacement occurs.

### DESCRIPTION OF EMBODIMENTS

The present invention will be described with reference to the drawings. The measurement of oxygen saturation will be described hereinbelow. But biofunctional information to be measured with the photoacoustic imaging apparatus of the present invention is not limited to oxygen saturation, and the total amount of hemoglobin or the like may also be measured. As long as biofunctional information inside a subject can be derived by irradiating the subject with at least two or more lights having different wavelengths to detect the difference between acoustic waves generated within the subject, the biofunctional information acquirement (photoacoustic imaging apparatus) of the present invention can be used for the measurement of any biofunctional information.

The present invention is not limited to a single apparatus having the following configuration. The present invention is also implemented by the use of a method for implementing functions described in this embodiment, and by processing in which software (computer program) for implementing these functions is supplied to one of a system and an apparatus via one of a network and various storage media, and the computer (or one of CPU, MPU, and the like) of one of the system and the apparatus reads and executes the program.

### Embodiment 1

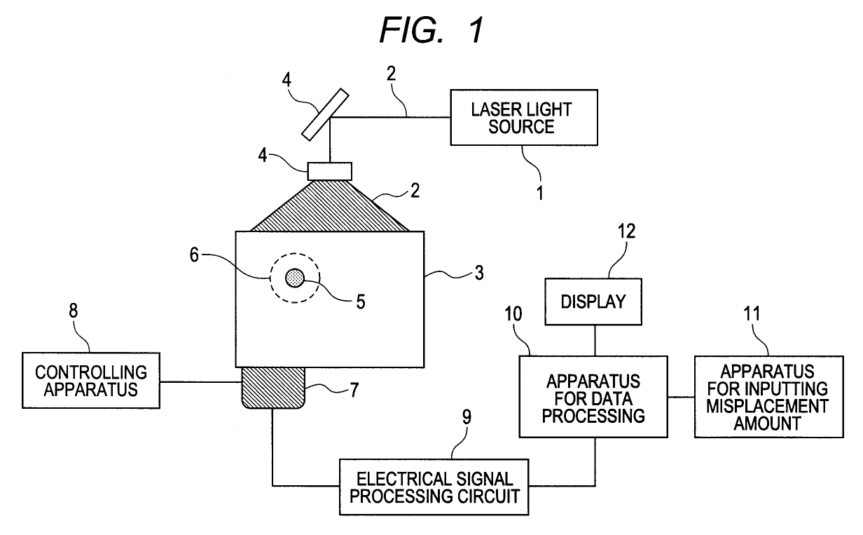

FIG. 1 illustrates a first embodiment of the photoacoustic imaging of the present invention. An exemplary mode for carrying out the present invention will be described based on FIG. 1.

An photoacoustic imaging apparatus in this embodiment includes a light source 1 which irradiates a subject 3 with light 2 having a single wavelength, optical devices 4, such as lenses, which guides the light 2 from the light source 1 to the subject 3, an acoustic detector 7 which detects an acoustic wave 6 generated when an optical absorber 5 absorbs the energy of the light propagated and diffused inside the subject 3, and converts the acoustic wave 6 to an electrical signal, a controlling apparatus 8 which allows the acoustic detector 7 to scan, an electrical signal processing circuit 9 which performs the amplification, digital conversion, and the like of the electrical signal, an apparatus 10 for data processing which constructs an image regarding in vivo information (generates image data), an apparatus 11 for inputting misplacement amount which inputs the position displacement amount of the subject, and a display 12 which displays the image. The light source 1 can output the lights 2 in at least two or more wavelengths.

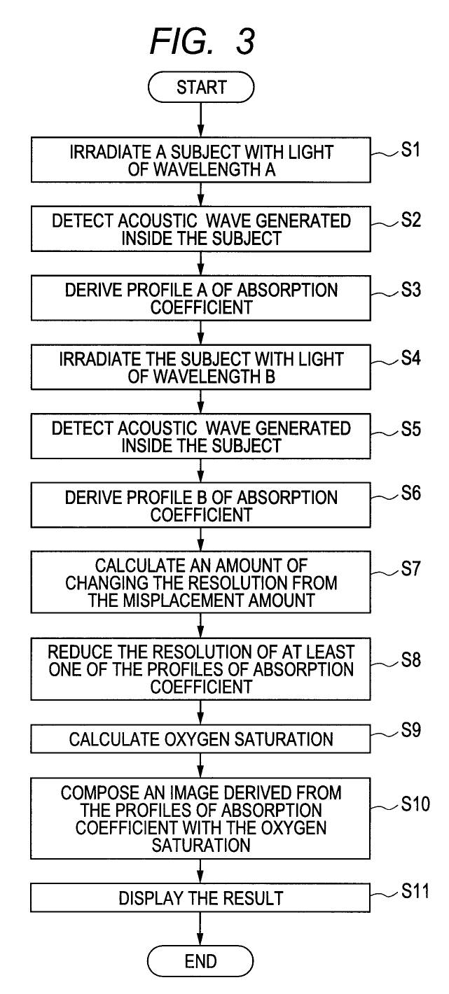

An implementation method will be described with reference to FIG. 1 and FIG. 3. The light 2 having a wavelength A (first wavelength) is pulsed, and the subject is irradiated with the pulsed light 2 (S1). When this light 2 is propagated and diffused inside the subject and absorbed by an optical absorber 5, the temperature of the absorber increases due to the absorption of the pulsed light. A volume expansion of the absorber occurs due to the temperature increase, and thus, an acoustic wave 6 is excited from the optical absorber 5. The generated acoustic wave 6 is received by an acoustic detector 7 acoustically coupled to the subject, and is converted to an electrical signal (S2). An acoustic wave detector may be acoustically coupled to the subject, and a shape retention member, such as a compression plate which constantly keeps the shape of the subject, may be provided between the subject and the acoustic wave detector. The acoustic detector 7 can be controlled by the controlling apparatus 8, and can measure the acoustic wave 6 in various places, while mechanically moving on a surface of the subject. More than two acoustic detectors may be simultaneously used in detecting an acoustic wave generated in a single irradiation. The detected electrical signal is converted to a digital signal by the electrical signal processing circuit 9, such as an amplifier and an analog-to-digital converter, and then reconstructed for profile A of absorption coefficient (profile of first absorption coefficient) of light having the wavelength A within the subject at a site of the subject irradiated with the light, by the apparatus 10 for data processing, such as a PC (S3). The above operations are also performed for a case where light having a wavelength B (second wavelength) is used, to derive profile B of absorption coefficient (profile of second absorption coefficient) of the wavelength B within the subject at the site of the subject irradiated with the light (S4 to S6). Further, as described later, internal processing to calculate oxygen saturation is performed in the apparatus 10 for data processing, which is based on the value of the position displacement between the position of the optical absorber 5 in the irradiation with the light having wavelength A and the position of the optical absorber 5 in the irradiation with the light having wavelength B, which positions being input to the apparatus 11 for inputting misplacement amount (S7 to S9). Profiles C, D, and so on of absorption coefficients may also be calculated using more lights having different wavelengths C, D, and so on, to thereby derive oxygen saturation using the profiles C, D, and so on of absorption coefficient. Finally, the derived oxygen saturation is superimposed on the profiles of absorption coefficient (S10), and the result is displayed on the display 12 (S11).

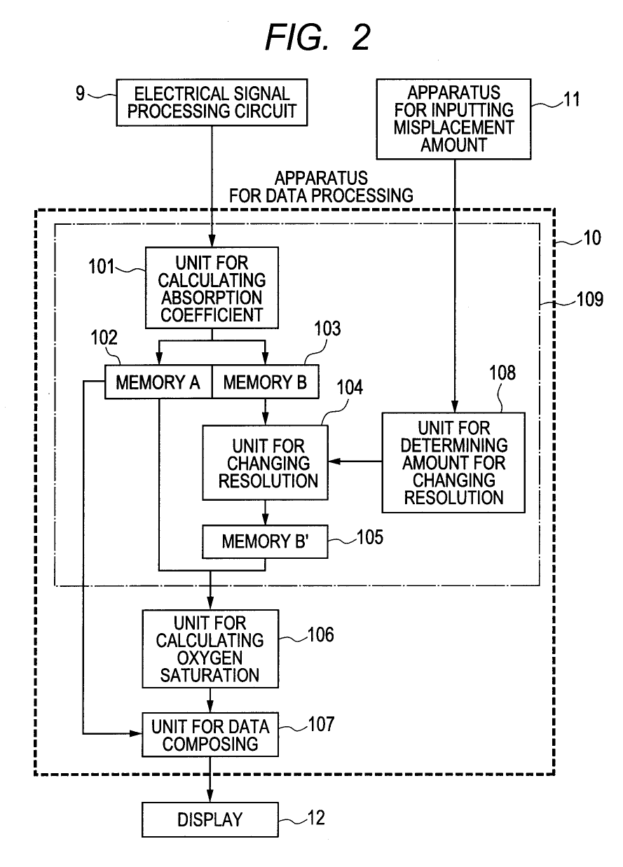

FIG. 2 and FIG. 3 show the internal processing of the apparatus 10 for data processing for carrying out the present invention. The apparatus for data processing 10 includes a unit 109 (first unit) for deriving an absorption coefficient, a unit 106 for calculating oxygen saturation as a unit for deriving biofunctional information (second unit), and a unit 107 (sixth unit/tenth unit) for composing. The unit 109 includes a unit 101 for calculating the absorption coefficient (third unit/eighth unit), a unit 104 for changing resolution (fourth unit/seventh unit), and a unit 108 for determining an amount for changing the resolution (fifth unit/ninth unit).

First, in a measurement using light having wavelength A, data showing profile A of absorption coefficient is calculated in the unit 101 by reconstructing the digital signal which is sent from the electrical signal processing circuit 9 (S3), and the calculated data showing the profile A of absorption coefficient (first data) is stored in a memory A102. In addition, for a measurement using light having wavelength B, similarly, data showing profile B of absorption coefficient (third data) is calculated (S6) and stored in a memory B103. Next, the position displacement amount between the position of the optical absorber 5 in the measurement using the light having the wavelength A and the position of the optical absorber 5 in the measurement using the light having the wavelength B is input to the apparatus 11 for inputting misplacement amount, and an amount for changing resolution is determined in the unit 108 based on the value of position displacement (S7). The unit 104 reduces image spatial resolution, by the determined amount for changing resolution, in at least one data among the data showing the profiles of absorption coefficient stored in the memories, to thereby derive a profile of absorption coefficient after the reduction (S8). In this invention, the data to which the image spatial resolution is reduced (second data) is used in calculating information on the subject such as oxygen saturation.

Although in this specification the present invention is explained mainly in a three-dimensional data processing, the invention can be applied to both of two-dimensional image data (pixel data) and three-dimensional image data (voxel data). Image spatial resolution in this invention is resolution in an image space, rather than resolution determined by the size of the element of the acoustic detector 7. In this Specification, spatial resolution in three-dimensional image data is referred to as voxel spatial resolution, and spatial resolution in two-dimensional image data is referred to as pixel spatial resolution. In addition, voxel spatial resolution and pixel spatial resolution are together defined as image spatial resolution. In FIG. 2, the image spatial resolution of the data showing the profile B of absorption coefficient (the profile of absorption coefficient that can be calculated in the irradiation with the light having the wavelength B) stored in the memory B103 is reduced. However the image spatial resolution of any of the data among the plurality of profiles of absorption coefficient may be reduced. In addition, while in FIG. 2 only an image spatial resolution of a single profile of absorption coefficient is reduced, more than two profiles of absorption coefficient may also be reduced.

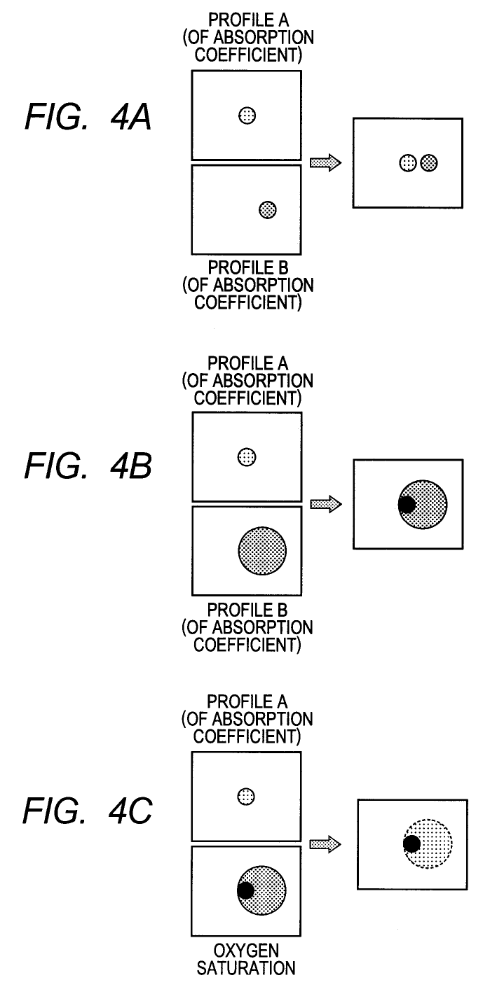

When position displacement occurs between the measurement using the light having the wavelength A and the measurement using the light having the wavelength B, usually, images of the same optical absorber cannot be compared, as illustrated in FIG. 4A, and a correct oxygen saturation cannot be derived. however, when the image spatial resolution is reduced to apparently increase the size of at least one image of the optical absorber (that is, increase the number of voxels corresponding to the optical absorber), a portion where both images of the optical absorber are composed is created as illustrated in FIG. 4B, and therefore, misdirected comparison operation between the value of the optical absorber and the value of a place other than the optical absorber due to the position displacement can be avoided. In other words, in FIG. 2 and FIG. 3, with reducing the image spatial resolution of the data showing the profile B of absorption coefficient, the image of the optical absorber in the data showing the profile A of absorption coefficient is included in the image of the optical absorber in the data showing the profile B of absorption coefficient of which the resolution is reduced.

At this time, since the oxygen saturation is calculated from the image whose resolution is changed, oxygen saturation in a wide region including the periphery of the optical absorber is derived. However, in the imaging of biofunctional information such as oxygen saturation, since (the absolute value of) a derived value shows the benignancy/malignancy of a tumor or the like, a quantitativeness of how much each of a plurality of lights is absorbed is of more importance than its resolution (which is important in the imaging of shape information such as a blood vessel image) of the image. Therefore, even if the oxygen saturation is derived by lowering the resolution and it is an average value of oxygen saturation in the image of the optical absorber (the image of the optical absorber in the profile of absorption coefficient before the resolution is reduced) and of oxygen saturation in the periphery of the image of the optical absorber, the utility value of the derived oxygen saturation is still large. In addition, in the present invention, the place where the optical absorber is actually present can be identified (that is, the resolution can be increased) in a subsequent step (S10). Therefore, even if the oxygen saturation is calculated at the cost of the resolution at this stage, the utility value is large as long as the quantitativeness is sufficiently high.

The amount for changing (the extent of reducing) the image spatial resolution at this time is determined according to a position displacement amount input to the apparatus 11 for inputting misplacement amount or a method used for the resolution reduction processing. In an elastic object such as a living body, even if a position displacement of a certain particular place is accurately grasped, the same amount of position displacement cannot be always applied to other places. Therefore, when an attempt is made to accurately align images by position adjustment, enormous measurements of a position displacement amount for voxels would become necessary. However, in the present invention, the portion where the images of the optical absorber are composed is created by reducing the image spatial resolution, and therefore, it is not necessary to grasp a position displacement amount for each voxel. However, in order to create the portion where the images of the optical absorber are composed, the image of the optical absorber after the reduction of the image spatial resolution must be enlarged in an amount more than the actual position displacement. Thus, while the position displacement amount input to the apparatus 11 for inputting misplacement amount may be a rough amount, a value certainly larger than the actual position displacement amount is used.

The amount for changing the image spatial resolution with respect to the position displacement amount is determined so that the image of the optical absorber in the profile of absorption coefficient whose resolution is not reduced is at least included (when the resolution of all profiles of absorption coefficient is reduced, any one profile of absorption coefficient before resolution is reduced is included) in the region of the image of the optical absorber in the profile of absorption coefficient after the reduction, regardless of the number of profiles of absorption coefficient whose image spatial resolution is changed. At this time, the amount for changing the image spatial resolution may be independently determined for each profile of absorption coefficient whose image spatial resolution is to be reduced, or the amount for changing the image spatial resolution may be equally determined for all profiles of absorption coefficient whose image spatial resolution is to be reduced. The method for deriving the position displacement amount is not particularly limited, and the position displacement amount can be derived with any publicly known method. The position displacement amount may be derived from mechanical measurement or measurement from images, and the input may be either manual or automatic. The amount of image spatial resolution to be changed with respect to the position displacement amount is different for each method for changing resolution. Therefore, the relationship between the position displacement amount and the amount for changing resolution may be previously obtained for each method for changing resolution and prepared as a table or a relation, and the amount for changing resolution may be determined using this previously prepared table or relation.

The method for reducing the image spatial resolution is not limited, and the reduction of the image spatial resolution can be achieved, for example, by the convolution of a spatial filter such as a digital filter. In this method, the calculation amount is not large, and practically extendable to three dimensions. As the filter, a filter that reduces resolution such as a moving average filter or a gaussian filter is used. The size of the image of the optical absorber in voxel data can be adjusted by changing the size of the filter. At this time, it is necessary to perform adjustment so that the images of the optical absorber overlap each other, as illustrated in FIG. 4B. Thus, in the unit 108, the position displacement amount between the images of the optical absorber is measured, and the amount for changing the size of the filter to overlap each other the images of the optical absorber is determined for each type of the filter, based on the measured position displacement amount between the images of the optical absorber.

The profile of absorption coefficient subjected to the resolution reduction processing is stored in a temporary memory B'105. When the resolution of the plurality of profiles of absorption coefficient is reduced, each profile of absorption coefficient is stored in a different temporary memory. Next, in the unit 106 for calculating oxygen saturation as a unit for calculating biofunctional information, oxygen saturation is derived using at least a profile of absorption coefficient whose resolution is reduced (S9). At this time, the profile of absorption coefficient whose image spatial resolution is reduced is used for at least one of the plurality of profiles of absorption coefficient used for obtaining the oxygen saturation. As long as at least one or more of the profile of absorption coefficient whose image spatial resolution is reduced are used, the oxygen saturation may be obtained using two or more profiles of absorption coefficient whose image spatial resolutions are reduced, or all profiles of absorption coefficient used may be the ones whose image spatial resolution is reduced. However, also here, the image of the optical absorber in the profile of absorption coefficient whose resolution is not reduced should be included in the region of the image of the optical absorber in the profile of absorption coefficient whose resolution is reduced. The method for calculating oxygen saturation will be described later.

Since the profile of absorption coefficient whose resolution is reduced is used, the derived oxygen saturation is the value of a region including the periphery of the image of the optical absorber. Therefore, in the unit 107, the derived information on the subject (e.g. oxygen saturation) is composed with the profile of absorption coefficient whose image spatial resolution is not reduced, as illustrated in FIG. 4C, and only the region of the image of the optical absorber (the image of the optical absorber in the case where the resolution is not reduced) is extracted (S10). In FIG. 2, it is possible to use the data showing the profile A of absorption coefficient whose resolution is not reduced for the profile of absorption coefficient used for the composing. Alternatively, it is possible to store another data showing the profile B of absorption coefficient of which the image spatial resolution is not reduced, and perform composing using the stored data showing the profile B of absorption coefficient. In addition, it is possible to perform composing, using data (fourth data) showing the profile of absorption coefficient (third absorption coefficient) of the wavelength C (third wavelength), not the wavelength A or the wavelength B.

The method for extracting only the region of the image of the optical absorber is not particularly limited. For example, only the portion of the optical absorber can be extracted in the profile of absorption coefficient whose image spatial resolution is not reduced, by previously determining the threshold value of a voxel which represents an absorption coefficient of the position where the optical absorber is present and performing threshold processing. In other words, only the portion of the optical absorber can be extracted, by substituting the value of oxygen saturation of a spatial coordinates only into the same voxel having a value equal to or more than a predetermined threshold in the profile of absorption coefficient whose image spatial resolution is not reduced, and making oxygen saturation zero in a portion with a value lower than the threshold in the profile of absorption coefficient whose image spatial resolution is not reduced. Also in two-dimensional data, only the portion of the optical absorber can be extracted, by substituting the value of oxygen saturation in a spatial coordinates only into the same pixel having a value equal to or more than a threshold in the profile of absorption coefficient whose pixel spatial resolution is not reduced.

At this time, it is also possible to also simultaneously extract a profile of absorption coefficient whose image spatial resolution for the position of the optical absorber is not changed and allow the value of oxygen saturation and the value of the profile of absorption coefficient to correspond to at least one color attribute, different from each other, of hue, saturation and lightness to derive spatial data (image data). For example, it is possible to determine hue by the value of oxygen saturation and determine saturation by the value of the profile of absorption coefficient for each voxel to perform drawing.

This result is displayed by the display 12 (S11).

Next, the method for calculating oxygen saturation will be described. When the main optical absorbers are deoxyhemoglobin and oxyhemoglobin, an absorption coefficient μₐ (λ) derived by measurement using light having a wavelength λ is the sum of the product of the absorption coefficient μ_{Hb} (λ) of deoxyhemoglobin and the abundance ratio C_{Hb} of deoxyhemoglobin, and the product of the absorption coefficient μ_{HbO2} (λ) of oxyhemoglobin and the abundance ratio C_{HbO2} of oxyhemoglobin, as shown in a formula (1). μ_{Hb} (λ) and μ_{HbO2} (λ) are physical properties with a determined value, and previously measured by other methods. The unknowns in the formula (1) are two, C_{Hb} and C_{HbO2}. Therefore, by performing measurement at least twice, using lights having different wavelengths, a simultaneous equation can be solved to calculate C_{Hb} and C_{HbO2}. When more measurements are performed, C_{Hb} and C_{HbO2} can be derived, for example, by fitting using the method of least squares.

μₐ(λ)=C_{Hb}·μ_{Hb}(λ)+C_{HbO}_{₂}·μ_{HbO}_{₂}(λ) (1)

Oxygen saturation SO₂ is the ratio of oxyhemoglobin in total hemoglobin and therefore calculated by a formula (2).

$${SO}_{2} = \frac{{C}_{{HbO}_{2}}}{{C}_{Hb} + {C}_{{HbO}_{2}}} \qquad ( 2 )$$

### Embodiment 2

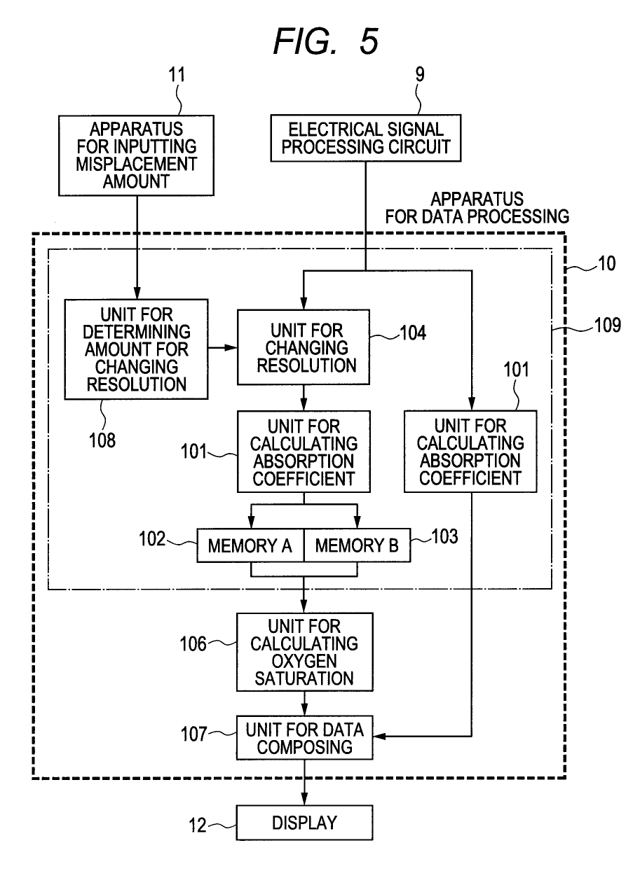

A method for placing band limitation on a signal derived by the acoustic wave detector, as a measure replacing the spatial filter described in Embodiment 1, to reduce the image spatial resolution of a derived profile of absorption coefficient and obtain the second data will be described using FIG. 2 and FIG. 5.

The internal processing of the apparatus 10 for data processing for carrying out the present invention, which is a differential point, will be described, and the remaining apparatus configuration is similar to the apparatus configuration of Embodiment 1. The profile A of absorption coefficient is calculated in the unit 101, using a digital signal which is sent from the electrical signal processing circuit 9 and which is obtained in measurement using the wavelength A. On the other hand, the amount for changing the resolution of a digital signal to be reduced is determined in the unit 108, based on a value derived from the apparatus 11 for inputting misplacement amount. The amount for changing the resolution is determined as in Embodiment 1. The amount of the resolution of the digital signal to be changed with respect to the misplacement amount is different for each method for changing the resolution. Therefore, the relationship between the position displacement amount and the amount for changing resolution may be previously obtained for each method for changing resolution and prepared as a table or a relation, and the amount for changing resolution may be determined using this previously prepared table or relation.

The image spatial resolution of the derived profile of absorption coefficient is reduced by processing a time-series digital signal which is sent from the electrical signal processing circuit 9 in the unit 104. In the unit 104, the resolution of the signal is reduced according to the amount for changing the resolution to derive a reduced signal (first reduced signal). In other words, resolution of a signal corresponding to light having at least one wavelength, among signals corresponding to lights having a plurality of wavelengths, is reduced more than resolution of other signals corresponding to light having a wavelength different from the at least one wavelength, to derive a reduced signal corresponding to light having the at least one wavelength. Specifically, for example, images of the optical absorber whose image spatial resolution is reduced by limiting the band of a signal are superimposed on each other. Alternatively, a reduced signal can also be calculated by summing signals of the acoustic detector derived at a plurality of positions and using the summed signals as a signal at one place, and image spatial resolution can be reduced. In the above processing methods, only signal processing may be performed on the time-series signal, and processing in a three-dimensional space is not necessary. Therefore, the processing amount in the entire process is small. The amount of the resolution of the digital signal to be changed with respect to the position displacement amount is different for each method for changing the resolution. Therefore, the relationship between the position displacement amount and the amount for changing resolution may be previously obtained for each method for changing resolution and prepared as a table or a relation, and the amount for changing resolution may be determined using this previously prepared table or relation. By calculating data showing a profile of absorption coefficient in the unit 101 using the processed signal, data showing a profile of absorption coefficient, whose resolution is reduced compared with data showing a profile of absorption coefficient calculated when using the signal before the processing, is derived. As in Embodiment 1, profiles of absorption coefficient in which resolution for at least one of measurements using the light having the wavelength A and wavelength B is reduced by the above mentioned method are calculated, data showing the calculated profiles of absorption coefficient is stored in the memory A102 and the memory B103, and the average intensity of oxygen saturation is calculated in the unit 106 for calculating oxygen saturation, using both of the data showing the calculated profiles of absorption coefficient. It is also possible to calculate oxygen saturation using more lights having different wavelengths C, D, and so on. Processing thereof is also similar to the processing of Embodiment 1. Next, the data showing the profile of absorption coefficient whose image spatial resolution is not reduced, and the intensity of oxygen saturation are composed in the unit 107, and the result is displayed on the display 12.

### Example 1

The calculation of oxygen saturation was simulated for each of a case where the position displacement of an optical absorber between measurements using a plurality of lights did not occur, a case where the position displacement occurred and processing for the position displacement was not performed, and a case where the position displacement occurred and Embodiment 1 was carried out.

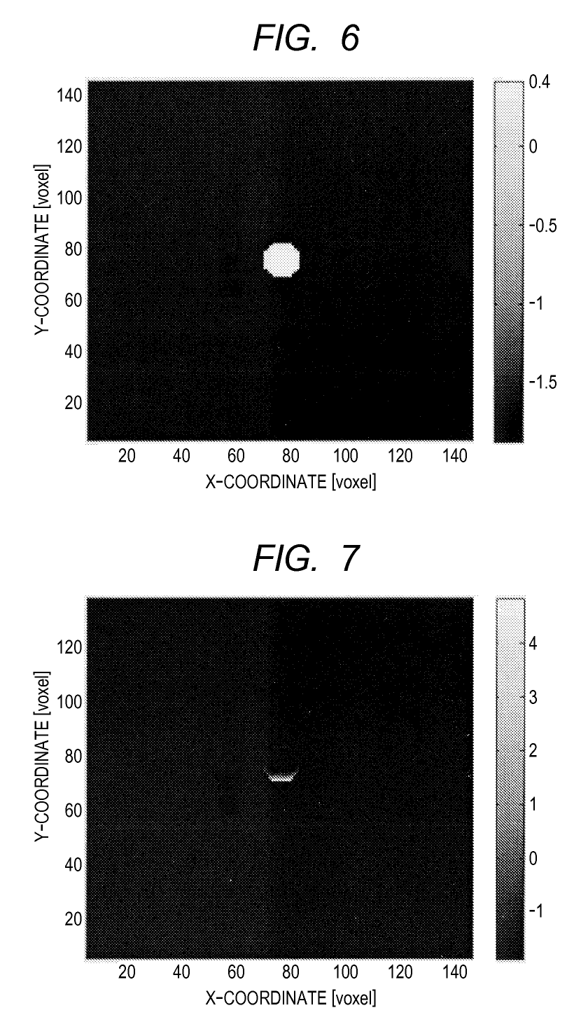

A spherical optical absorber having a diameter of 2 mm, in which 40% of oxyhemoglobin and 60% of deoxyhemoglobin were mixed to simulate blood, was placed at the center of a subject and irradiated with 800 nm and 850 nm lights, and signals thereof were derived by simulation. Profiles of absorption coefficient were respectively derived using both signals. Oxygen saturation was calculated without displacing both profiles of absorption coefficient, and is illustrated in FIG. 6. The concentration of the portion of the spherical optical absorber was 0.4, and the calculated oxygen saturation was 40%. In this manner, for the oxygen saturation when the position displacement did not occur, the concentration of oxyhemoglobin was calculated correctly.

For comparison, a case where the position displacement occurred and processing for the position displacement was not particularly performed will be described. When the profiles of absorption coefficient of 800 nm and 850 nm were vertically displaced by 2 mm, the oxygen saturation derived by a conventional method not reducing resolution was as illustrated in FIG. 7. In this manner, when the position displacement occurred, the oxygen saturation could not be correctly calculated.

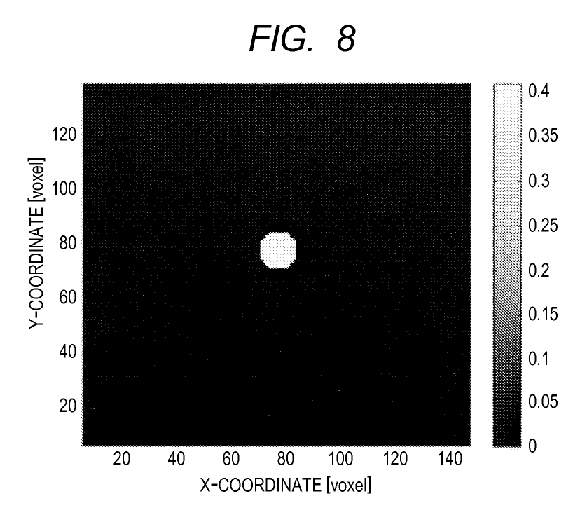

The result of carrying out the processing of Embodiment 1 when the position displacement occurred is illustrated in FIG. 8. Here, the voxel spatial resolution of the profiles of absorption coefficient of both 800 nm and 850 nm was reduced by a factor of 7 by the convolution of a moving average filter, and oxygen saturation was calculated using the results. Further, the calculated oxygen saturation was displayed for only voxels having a value equal to or more than 50% of the maximum value in the profile of absorption coefficient of 800 nm whose voxel spatial resolution was not reduced. As a result, the concentration of the portion of the spherical optical absorber was about 0.4, and the calculated oxygen saturation was 40%. It was shown that by using the present invention, oxygen saturation can be calculated with a minor error even if position displacement occurs. In addition, the increase in calculation time at this time was negligible compared with the conventional method.

### Example 2

An example will be described in which simulation similar to the simulation of Example 1 was performed and a method for summing acoustic signals derived at a plurality of positions and using the summed signals as a signal at one place was used as a method for reducing the voxel spatial resolution of a profile of absorption coefficient.

Acoustic signals generated by irradiating an optical absorber, in which 40% of oxyhemoglobin and 60% of deoxyhemoglobin were mixed, with 800 nm and 850 nm lights were derived by simulation. At this time, the probe for deriving acoustic signals included 100×100 square elements having a side of 2 mm, arrayed without gap in-between. Assuming that position displacement occurred during measurement for 800 nm and 850 nm, the position of the absorber was vertically displaced by 2 mm during the simulation for 800 nm and 850 nm.

For both of the 800 nm and 850 nm lights, signals of 5×5 elements were summed and regarded as a signal of one virtual element, and signals of 20×20 virtual elements were derived. Therefore, when the signals of the virtual elements were used, the voxel spatial resolution increased by a factor of 5, compared with a case where the absorption coefficient was calculated using each element. The profiles of absorption coefficient of 800 nm and 850 nm derived using the virtual elements were subjected to comparison operation to calculate oxygen saturation. The oxygen saturation of only voxels whose value was equal to or more than 50% of the maximum value in the profile of absorption coefficient of 800 nm before the signals derived using each element were summed was displayed. The displayed oxygen saturation of the voxels was about 40%. In this manner, even if position displacement occurred, images of the optical absorber were superimposed by processing the signals, and oxygen saturation was derived with a minor error. The increase in calculation time at this time was negligible, compared with the conventional method.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-022892, filed Feb. 4, 2010 and Japanese Patent Application No. 2011-010534, filed January 21, 2011, which are hereby incorporated by reference herein in their entirety.

### REFERENCE SIGNS LIST

1 light source

2 light

3 subject

4 optical device

5 optical absorber

6 acoustic wave

7 acoustic detector

8 controlling apparatus

9 electrical signal processing circuit

10 apparatus for data processing

11 apparatus for inputting misplacement amount

12 display

1. An apparatus for acquiring bio-functional information, comprising:
an acoustic wave detector, for receiving a plurality of acoustic waves generated when a subject is irradiated with a plurality of lights having different wavelengths, and for converting the plurality of acoustic waves to a plurality of signals corresponding to the plurality of lights; and
a processing apparatus for deriving bio-functional information inside the subject using a plurality of profiles of absorption coefficient which are derived from the plurality of signals and which correspond respectively to the plurality of signals,
wherein said processing apparatus comprises:
a first unit for deriving, from a signal corresponding to light having a first wavelength, first data showing a profile of first absorption coefficient corresponding to the light having the first wavelength, and deriving, from a signal corresponding to light having a second wavelength different from the first wavelength, second data showing a profile of second absorption coefficient corresponding to the light having the second wavelength; and
a second unit for deriving the bio-functional information using the first data and the second data, and
wherein the second data has lower image spatial resolution than the first data.

2. The apparatus for acquiring bio-functional information according to claim 1, wherein said first unit comprises:
a third unit for deriving the first data showing the profile of first absorption coefficient corresponding to the light having the first wavelength, and third data showing a profile of absorption coefficient corresponding to the light having the second wavelength; and
a fourth unit for reducing an image spatial resolution of the third data to derive the second data.

3. The apparatus for acquiring bio-functional information according to claim 2, wherein said first unit comprises a fifth unit for determining an amount for changing the image spatial resolution of the third data, so that when the image spatial resolution of the third data is reduced by that amount, a region corresponding to an image of an optical absorber in the first data is included in a region corresponding to an image of the optical absorber in the second data.

4. The apparatus for acquiring bio-functional information according to claim 3, wherein said fifth unit determines the amount for changing the image spatial resolution using a relationship between a position displacement amount and an amount for changing image spatial resolution, the relationship being previously prepared for each method for changing image spatial resolution.

5. The apparatus for acquiring bio-functional information according to claim 2, wherein said fourth unit reduces the image spatial resolution of the third data by convolution of a spatial filter.

6. The apparatus for acquiring bio-functional information according to claim 2, comprising a sixth unit for composing the bio-functional information derived by said second unit and one of the first data, the third data, and fourth data which is derived from a signal corresponding to light having a third wavelength and which shows a profile of third absorption coefficient corresponding to the light having the third wavelength, to derive only information on the subject in a region corresponding to an image of the optical absorber in the data whose image spatial resolution is not reduced.

7. The apparatus for acquiring bio-functional information according to claim 1, wherein said first unit comprises:
a seventh unit for reducing a resolution of the signal corresponding to the light having the second wavelength to be less than a resolution of the signal corresponding to the light having the first wavelength to acquire a first reduced signal corresponding to the light having the second wavelength, and
an eighth unit for deriving the first data showing the profile of first absorption coefficient corresponding to the light having the first wavelength from a signal corresponding to light having a first wavelength, and deriving the second data showing the profile of second absorption coefficient corresponding to the light having the second wavelength from the first reduced signal.

8. The apparatus for acquiring bio-functional information according to claim 7, wherein said first unit comprises a ninth unit for determining an amount for changing the resolution of the signal corresponding to the light having the second wavelength, so that when the resolution of the signal corresponding to the light having the second wavelength is reduced by that amount, a region corresponding to an image of an optical absorber in the first data will be included in an image of the optical absorber in the second data.

9. The apparatus for acquiring bio-functional information according to claim 8, wherein said ninth unit determines the amount for changing the resolution of the signal corresponding to the light having the second wavelength using a relationship between a position displacement amount and an amount for changing resolution, the relationship being previously prepared for each method for changing the resolution of the signal corresponding to the light having the second wavelength.

10. The apparatus for acquiring bio-functional information according to claim 7, wherein said seventh unit reduces the image spatial resolution of the signal corresponding to the light having the second wavelength by processing a time-series signal output from said acoustic wave detector.

11. The apparatus for acquiring bio-functional information according to claim 7, comprising a tenth unit for composing the bio-functional information derived by said second unit, and one of the first data and fourth data which is derived from a signal corresponding to light having a third wavelength and which shows a profile of third absorption coefficient corresponding to the light having the third wavelength, to derive only information on a subject in a region corresponding to an image of the optical absorber in the data whose image spatial resolution is not reduced.

12. The apparatus for acquiring bio-functional information according to claim 6, wherein said sixth unit performs, in the profile of absorption coefficient whose image spatial resolution is not reduced, processing for substituting a value of the bio-functional information in a spatial coordinate only into a pixel or a voxel of the same spatial coordinate having a value equal to or more than a predetermined threshold, and making a value of the bio-functional information zero in a spatial coordinate having a value lower than the threshold.

13. The apparatus for acquiring bio-functional information according to claim 6, wherein said sixth unit derives image data in which each of, a value of the bio-functional information and a value of the profile of absorption coefficient whose image spatial resolution is not changed, correspond to at least one color attribute, different from each other, of hue, saturation and lightness.

14. The apparatus for acquiring bio-functional information according to claim 1, wherein the plurality of signals corresponding to the plurality of lights include a plurality of signals which are derived by detecting and converting, an acoustic wave generated when the subject is irradiated with light having a wavelength, by using a plurality of acoustic detectors.

15. A method for acquiring bio-functional information by receiving acoustic waves generated when a subject is irradiated with a plurality of lights having different wavelengths, converting the acoustic waves to a plurality of signals corresponding to the plurality of lights, by an acoustic wave detector, and deriving bio-functional information using a plurality of profiles of absorption coefficient which are calculated from the plurality of signals and are respectively corresponding to the plurality of signals, comprising the steps of:
deriving, from an acoustic wave generated when the subject is irradiated with light having a first wavelength, first data showing a profile of first absorption coefficient corresponding to the light having the first wavelength;
deriving, from an acoustic wave generated when the subject is irradiated with light having a second wavelength, second data showing a profile of second absorption coefficient corresponding to the light having the second wavelength, and having lower image spatial resolution than the first data; and
deriving the bio-functional information using the first data and the second data.

16. A non-transitory computer-readable medium storing, in executable form, a program for allowing a computer to execute each step of a method for acquiring bio-functional information according to claim 14.

17. The apparatus for acquiring bio-functional information according to claim 11, wherein said tenth unit performs, in the profile of absorption coefficient whose image spatial resolution is not reduced, processing for substituting a value of the bio-functional information in a spatial coordinate only into a pixel or a voxel of the same spatial coordinate having a value equal to or more than a predetermined threshold, and making a value of the bio-functional information zero in a spatial coordinate having a value lower than the threshold.

18. The apparatus for acquiring bio-functional information according to claim 11, wherein said tenth unit derives image data in which each of, a value of the bio-functional information and a value of the profile of absorption coefficient whose image spatial resolution is not changed, correspond to at least one color attribute, different from each other, of hue, saturation and lightness.
